(19) United States
(12) Patent Application Publication
(10) Pub. No.: US 2008/0103557 A1
(43) Pub. Date: May 1, 2008

(54) Extended shelf life storable implantable medical device assembly, shipping container and method

(75) Inventors: Timothy J. Davis, Coon Rapids, MN (US); Alan Robert Helfinstine, Brooklyn Center, MN (US); Boysie R. Morgan, Minneapolis, MN (US); David P. Olson, Minnetrista, MN (US); Todd V. Smith, Shoreview, MN (US); Nathan A. Torgerson, Andover, MN (US); Leroy L. Perz, Buffalo, MN (US)

Correspondence Address: IPLM GROUP, P.A., POST OFFICE BOX 18455, MINNEAPOLIS, MN, 55418, US

(73) Assignee: Medtronic, Inc.

(21) Appl. No.: 11/590,684

(22) Filed: Oct. 31, 2006

(51) Int. Cl. A61N 1/00

(52) U.S. Cl. 607/61

(57) ABSTRACT

Storable implantable medical device assembly and container for an implantable device having a charging sub-assembly. The implantable medical device has therapeutic componentry and a rechargeable power source operatively coupled to the therapeutic componentry. The charging sub-assembly having an electro-chemical power source, such as a battery, and a charging circuit operatively coupled to the electro-chemical power source. The implantable medical device and the charging sub-assembly are co-located within the container. The charging circuit of the charging sub-assembly is operatively coupled to the chargeable power source within the container to charge the rechargeable power source while the implantable medical device remains in the container. The charging sub-assembly may use inductive coupling to charge the implantable device mimicking implantable device charging following implantation.

### FIELD OF THE INVENTION

This invention relates to implantable medical devices and, in particular, to shipping containers, methods and sub-assemblies.

### BACKGROUND OF THE INVENTION

Implantable medical devices for producing a therapeutic result in a patient are well known. Examples of such implantable medical devices include implantable drug infusion pumps, implantable neurostimulators, implantable cardioverters, implantable cardiac pacemakers, implantable defibrillators and cochlear implants. Of course, it is recognized that other implantable medical devices are envisioned which utilize energy delivered or transferred from an external device.

A common element in most of these implantable medical devices is the need for electrical power in the implanted medical device. The implanted medical device typically requires electrical power to perform its therapeutic function whether it be driving an electrical infusion pump, providing an electrical neurostimulation pulse or providing an electrical cardiac stimulation pulse. This electrical power is derived from a power source.

Typically, a power source for an implantable medical device is a rechargeable power source such as rechargeable batteries and, in particular, lithium ion batteries. Such batteries can have a capacity which is exhausted much earlier than the useful life of the implantable medical device. Electrical power can be transcutaneously transferred to the implanted medical device to recharge the rechargeable batteries through the use of inductive coupling. An external power source temporarily positioned on the surface of the skin of the patient can recharge the implanted medical device's batteries. In fact, many systems and methods have been used for transcutaneously inductively recharging a rechargeable power source used in an implantable medical device.

Transcutaneous energy transfer through the use of inductive coupling involves the placement of two coils positioned in close proximity to each other on opposite sides of the cutaneous boundary. The internal coil, or secondary coil, is part of or otherwise electrically associated with the implanted medical device. The external coil, or primary coil, is associated with the external power source or external charger, or recharger. The primary coil is driven with an alternating current. A current is induced in the secondary coil through inductive coupling. This current can then be used to charge, or recharge, an internal power source.

It is usually desirable for a rechargeable implantable medical device to be fully charged when such rechargeable implantable medical device is implanted into a patient. Some rechargeable implantable medical devices are transcutaneously charged or recharged after implantation through inductive coupling using an external charging device.

Inductive charging of the implanted medical device usually requires placing an external antenna directly on the skin of the patient at the site of implantation. However, the implantation site will typically still be recovering from the trauma of implantation immediately or soon after implantation. The implantation site can be adversely affected by the external charging unit if charging of the newly implanted medical device is attempted. Therefore, it is desirable to put off charging a newly implanted medical device for as long as possible, or typically one full charge period of the implanted medical device.

### BRIEF SUMMARY OF THE INVENTION

Implantable medical devices are commonly shipped from the manufacturer to a medical facility where implantation of the device is performed. Following shipment to a medical facility, such implantable medical devices can remain in storage until implantation occurs.

It is desirable for an implantable medical device to be fully charged at the time of implantation. This will allow a maximum amount of time for the implantation site to heal before transcutaneous charging is attempted.

If the implantable medical device is not fully charged at the time of shipment or on the inventory of a medical supply company, then the implantable medical device typically needs to be charged prior to implantation. Even if the implantable medical device is fully charged at the time of manufacture or at the time of shipment, the time period necessarily elapsing between manufacture and/or shipment to the time of implantation may lead the implantation medical device to lose some of its initial charge. Thus, in either case of pre-shipment fully charging or not, an implantable medical device may not be in fully charged condition when the implantable medical device is removed from its container just prior to implantation unless a separate charge or recharge event occurs just prior to implantation.

During shipment and storage, an implantable medical device is usually contained in a sterile environment in a storage container, such as a box. This would help to prevent contamination of the implantable medical device with germs, for example, and would help prevent subsequent infection of the patient upon implantation. Removal of the implantable medical device from the sterile environment of the shipping and/or storage container could subject the implantable medical device to the risk of contamination.

Thus, medical practitioners can be faced with a dilemma of removing the implantable medical device from the sterile environment of the container in order to charge or recharge the device and being able to implant a fully charged medical device, or ensuring that the implantable medical device remains sterile but only being able to implant a partially charged medical device which could limit early use of the medical device or subject the already traumatized implantation site to the additional trauma of an external antenna soon after implantation.

Aspects of the present invention provide a charging mechanism in the container in which the implantable medical device is stored during inventory, shipped or stored awaiting implant. Such a charging mechanism can provide occasional, periodic or continuous charging of a rechargeable power source of such an implantable medical device either preventing such power source from losing charge during inventory, shipment or storage or replenishing such charge during inventory, shipment or storage.

In an embodiment, the present invention provides a storable implantable medical device assembly having a container, an implantable device and a charging sub-assembly. The implantable medical device has therapeutic componentry, a rechargeable power source operatively coupled to the therapeutic componentry and a secondary coil operatively coupled to the rechargeable power source. The charging sub-assembly having an electro-chemical power source, such as a battery, a charging circuit operatively coupled to the electro-chemical power source and a primary coil operatively to the charging circuit. The implantable medical device and the charging sub-assembly are co-located within the container. The primary coil of the charging sub-assembly and the secondary coil of the implantable medical device being arranged in the container in juxtaposed relationship with the charging sub-assembly charging the rechargeable power source of the implantable medical device through inductive coupling between the primary coil of the charging sub-assembly and the secondary coil of the implantable medical device.

In an embodiment, the present invention provides a shipping container for an implantable medical device having therapeutic componentry, a rechargeable power source operatively coupled to the therapeutic componentry and a secondary coil operatively coupled to the rechargeable power source having a container and a charging sub-assembly having an electro-chemical power source, a charging circuit operatively coupled to the electro-chemical power source and a primary coil operatively coupled to the charging circuit. The container is arranged to be able to co-locate the implantable medical device and the charging sub-assembly within the container during shipment. The primary coil of the charging sub-assembly and the secondary coil of the implantable medical device, when so co-located, is arranged in the container in juxtaposed relationship with the charging sub-assembly charging the rechargeable power source of the implantable medical device through inductive coupling between the primary coil of the charging sub-assembly and the secondary coil of the implantable medical device.

In an embodiment, the implantable medical device is contained within a sterile sub-container within the container.

In an embodiment, the charging sub-assembly is located within a non-sterile area of the container.

In an embodiment, the battery comprises a disposable battery.

In an embodiment, the disposable battery is a plurality of alkaline batteries.

In an embodiment, the present invention provides a storable implantable medical device assembly having a container, an implantable device and a charging sub-assembly. The implantable medical device has therapeutic componentry and a rechargeable power source operatively coupled to the therapeutic componentry. The charging sub-assembly having an electro-chemical power source, such as a battery, and a charging circuit operatively coupled to the electro-chemical power source. The implantable medical device and the charging sub-assembly are co-located within the container. The charging circuit of the charging sub-assembly is operatively coupled to the chargeable power source within the container to charge the rechargeable power source while the implantable medical device remains in the container.

In an embodiment, the present invention provides a shipping container for an implantable medical device having therapeutic componentry and a rechargeable power source operatively coupled to the therapeutic componentry having a container and a charging sub-assembly having an electro-chemical power source and a charging circuit operatively coupled to the electro-chemical power source. The container is arranged to be able to co-locate the implantable medical device and the charging sub-assembly within the container during shipment. The charging circuit of the charging sub-assembly being operable to charge the rechargeable power source while the implantable medical device remains in the container.

In an embodiment, the charging circuit is directly connected to the rechargeable power source through a hard-wired connection.

In an embodiment, the present invention provides a method of storing an implantable medical device having therapeutic componentry and a rechargeable power source operatively coupled to the therapeutic componentry and a rechargeable power source operatively coupled to the therapeutic componentry. The implantable medical device and the charging sub-assembly are co-located in a single container. The rechargeable power source of the implantable medical device is operatively charged with the charging sub-assembly while located in the container.

In an embodiment, the charging sub-assembly contains an electro-chemical power source as a source of power to charge the rechargeable power source of the implantable medical device.

In an embodiment, the implantable medical device is shipped in the container while the rechargeable power source is being recharged by the charging sub-assembly.

In an embodiment, the charging sub-assembly is disposed of following shipment.

In an embodiment, operative coupling of the charging sub-assembly and the rechargeable power source of the implantable medical device is hard-wired.

In an embodiment, the implantable medical device further has a secondary coil operatively coupled to the rechargeable power source, the charging sub-assembly further has a secondary coil and the primary coil of the charging sub-assembly is co-located in a juxtaposed relationship with the secondary coil of the implantable medical device.

In an embodiment, the rechargeable power source of the implantable medical device is recharged through inductive coupling between the primary coil of the charging sub-assembly and the secondary coil of the implantable medical device.

### BRIEF DESCRIPTION OF THE DRAWINGS

FIG. 1 illustrates an implantable medical device implanted in a patient;

FIG. 2 is a block diagram of an implantable medical device;

FIG. 3 is a detailed block diagram of an implantable medical device implanted subcutaneously and an associated external charging device in accordance with an embodiment of the present invention;

FIG. 4 is a diagrammatic block diagram of a storable implantable medical device assembly and shipping container having a disposable charger;

FIG. 5 is a block diagram of an embodiment of a storable implantable medical device assembly and shipping container having a disposable charger in a sterile environment;

FIG. 6 is a block diagram of an embodiment of a storable implantable medical device assembly and shipping container having a disposable charger with an implantable medical device in a sterile environment with a disposable charger in a non-sterile environment; and

FIG. 7 is a block diagram of an embodiment of a storable implantable medical device assembly and shipping container having a disposable charger utilizing a direct connection for charging.

### DETAILED DESCRIPTION OF THE INVENTION

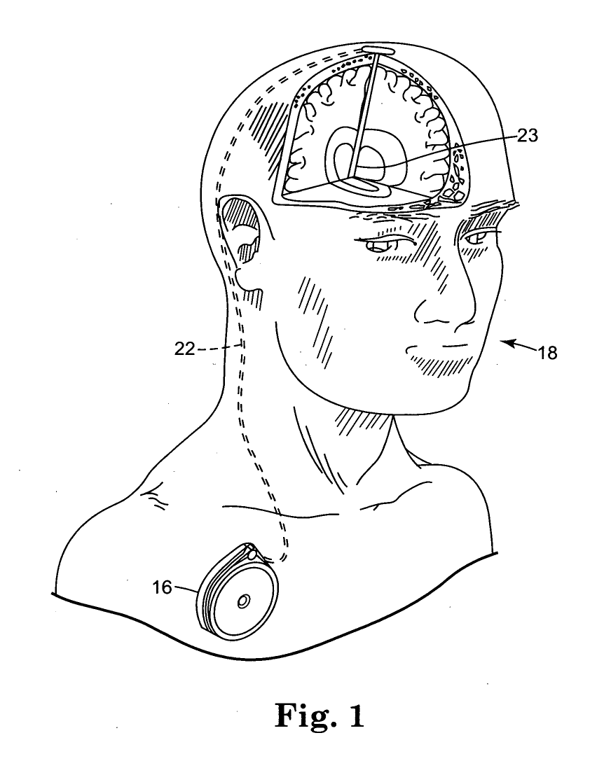

FIG. 1 shows implantable medical device 16, for example, a drug pump, implanted in patient 18. The implantable medical device 16 is typically implanted by a surgeon in a sterile surgical procedure performed under local, regional, or general anesthesia. Before implanting the medical device 16, a catheter 22 is typically implanted with the distal end position at a desired therapeutic delivery site 23 and the proximal end tunneled under the skin to the location where the medical device 16 is to be implanted. Implantable medical device 16 is generally implanted subcutaneously at depths, depending upon application and device 16, of from 1 centimeter (0.4 inches) to 2.5 centimeters (1 inch) where there is sufficient tissue to support the implanted system. Once medical device 16 is implanted into the patient 18, the incision can be sutured closed and medical device 16 can begin operation.

Implantable medical device 16 operates to infuse a therapeutic substance into patient 18. Implantable medical device 16 can be used for a wide variety of therapies such as pain, spasticity, cancer, and many other medical conditions.

The therapeutic substance contained in implantable medical device 16 is a substance intended to have a therapeutic effect such as pharmaceutical compositions, genetic materials, biologics, and other substances. Pharmaceutical compositions are chemical formulations intended to have a therapeutic effect such as intrathecal antispasmodics, pain medications, chemotherapeutic agents, and the like. Pharmaceutical compositions are often configured to function in an implanted environment with characteristics such as stability at body temperature to retain therapeutic qualities, concentration to reduce the frequency of replenishment, and the like. Genetic materials are substances intended to have a direct or indirect genetic therapeutic effect such as genetic vectors, genetic regulator elements, genetic structural elements, DNA, and the like. Biologics are substances that are living matter or derived from living matter intended to have a therapeutic effect such as stem cells, platelets, hormones, biologically produced chemicals, and the like. Other substances may or may not be intended to have a therapeutic effect and are not easily classified such as saline solution, fluoroscopy agents, disease diagnostic agents and the like. Unless otherwise noted in the following paragraphs, a drug is synonymous with any therapeutic, diagnostic, or other substance that is delivered by the implantable infusion device.

Implantable medical device 16 can be any of a number of medical devices such as an implantable therapeutic substance delivery device, implantable drug pump, cardiac pacemaker, cardioverter or defibrillator, as examples.

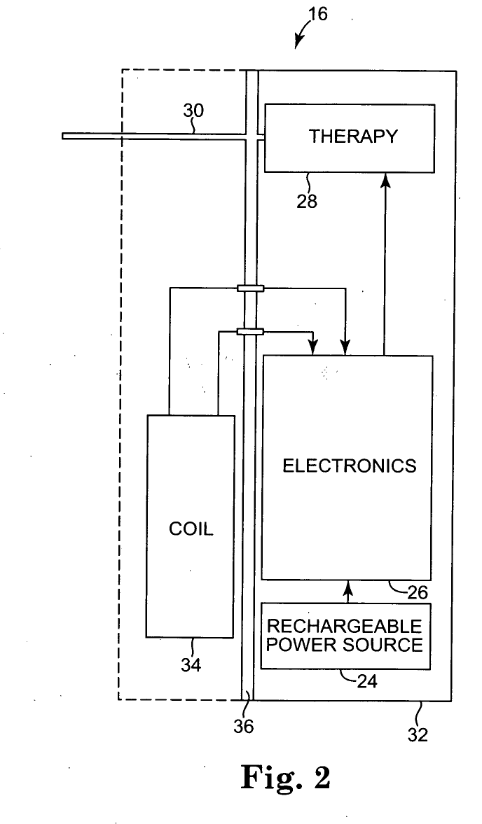

In FIG. 2, implantable medical device 16 has a rechargeable power source 24, such as a Lithium ion battery, powering electronics 26 and therapy module 28 in a conventional manner. Therapy module 28 is coupled to patient 18 through one or more therapy connections 30, also conventionally. Rechargeable power source 24, electronics 26 and therapy module 28 are contained in hermetically sealed housing 32. Secondary charging coil 34 may be attached to the exterior of housing 32. Secondary charging coil 34 is operatively coupled through electronics 26 to rechargeable power source 24. In an alternative embodiment, secondary charging coil 34 could be contained in housing 32 or could be contained in a separate housing umbilically connected to electronics 26. Electronics 26 help provide control of the charging rate of rechargeable power source 24 in a conventional manner. Magnetic shield 36 is optionally positioned between secondary charging coil 34 and housing 32 in order to protect rechargeable power source 24, electronics 26 and therapy module 28 from electromagnetic energy when secondary charging coil 34 is utilized to charge rechargeable power source 24.

Rechargeable power source 24 can be any of a variety power sources including a chemically based battery or a capacitor. In a preferred embodiment, rechargeable power source is a well known lithium ion battery.

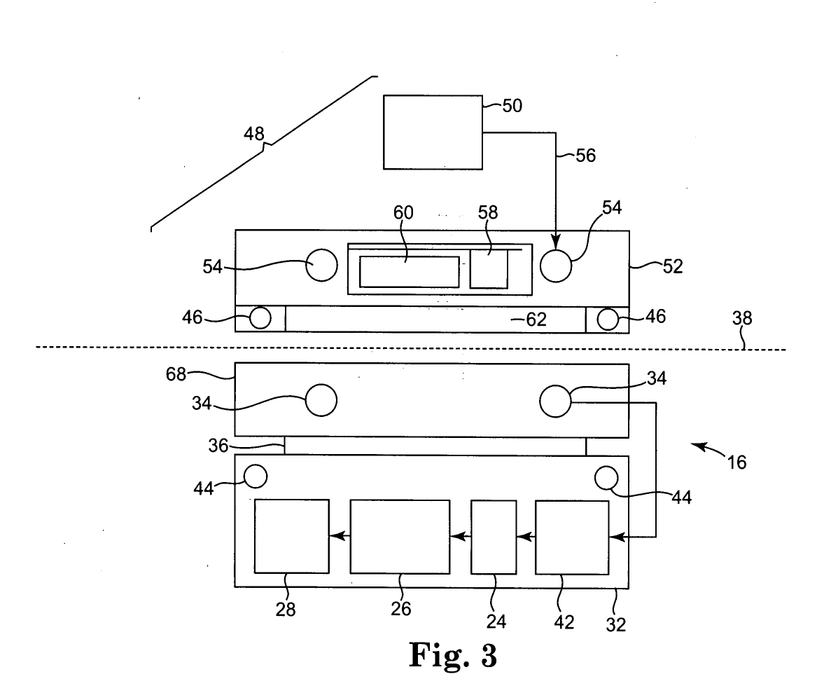

FIG. 3 illustrates an alternative embodiment of implantable medical device 16 situated under cutaneous boundary 38. Implantable medical device 16 is similar to the embodiment illustrated in FIG. 2. However, charging regulation module 42 is shown separate from electronics 26 controlling therapy module 28. Again, charging regulation and therapy control is conventional. Implantable medical device 16 also has internal telemetry coil 44 configured in conventional manner to communicate through external telemetry coil 46 to an external programming device (not shown), charging unit 50 or other device in a conventional manner in order to both program and control implantable medical device and to externally obtain information from implantable medical device 16 once implantable medical device has been implanted. Internal telemetry coil 44 is sized to be larger than the diameter of secondary charging coil 34. Magnetic shield 36 is optionally positioned between secondary charging coil 34 and housing 32 and sized to cover the footprint of secondary charging coil 34.

Internal telemetry coil 44, having a larger diameter than secondary coil 34, is not completely covered by magnetic shield 36 allowing implantable medical device 16 to communicate with the external programming device with internal telemetry coil 44 in spite of the presence of magnetic shield 36.

Rechargeable power source 24 can be charged while implantable medical device 16 is in place in a patient through the use of external charging device 48. In a preferred embodiment, external charging device 48 consists of charging unit 50 and external antenna 52. Charging unit 50 contains the electronics necessary to drive primary coil 54 with an oscillating current in order to induce current in secondary coil 34 when primary coil 54 is placed in the proximity of secondary coil 34. Charging unit 50 is operatively coupled to primary coil by cable 56. In an alternative embodiment, charging unit 50 and antenna 52 may be combined into a single unit. Antenna 52 may also optionally contain external telemetry coil 46 which may be operatively coupled to charging unit 50 if it is desired to communicate to or from implantable medical device 16 with external charging device 48. Alternatively, antenna 52 may optionally contain external telemetry coil 46 which can be operatively coupled to an external programming device, either individually or together with external charging unit 48.

Inductive coupling between primary coil 54 of external antenna 52 and secondary coil of implantable medical device 16 is accomplished at a drive, or carrier, frequency, f_{carrier}, in the range of from eight (8) to twelve (12) kiloHertz. In a preferred embodiment, the carrier frequency f_{carrier}, of external antenna 54 is approximately nine (9) kiloHertz unloaded.

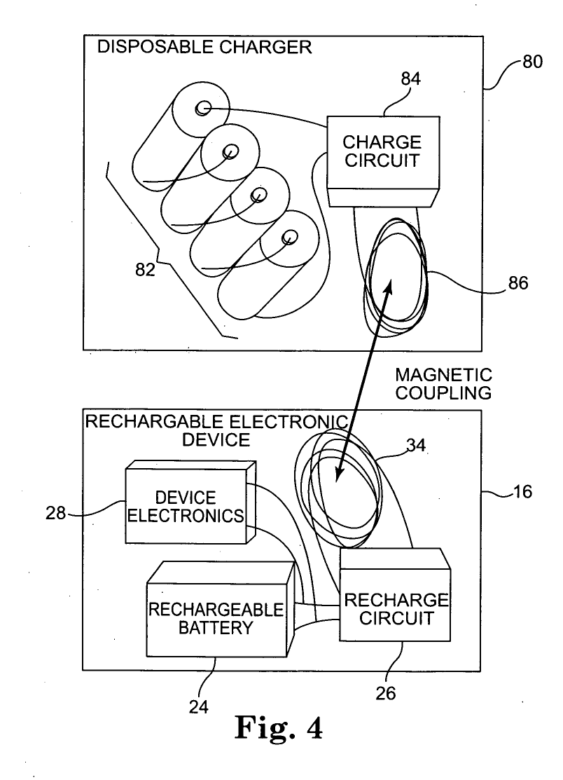

Following manufacture and before implantation, for example, during inventory storage, during shipment and/or while awaiting implantation, implantable medical device 16 may be stored in along with charging sub-assembly 80, illustrated along with implantable medical device 16 in FIG. 4. Implantable medical device 16 contains the customary componentry associated with a rechargeable implantable medical device 16 including therapeutic componentry 28 powered from rechargeable power source 24, such as a rechargeable battery. Rechargeable power source 24 is coupled to recharge circuitry 26 for conventionally controlling the charge level of rechargeable power source 24 and secondary coil 34 adapted to receive energy through inductive coupling from an external power source. Packaged with implantable medical device 16 is charging sub-assembly 80 used to either maintain the charge level, e.g., fully charged, of rechargeable power source 24 of implantable medical device 16 or to replenish the charge level, again e.g., fully charged, of rechargeable power source 24 of implantable medical device 16.

Charging sub-assembly 80 contains an electro-chemical power source 82, charge circuit 84 and primary coil 86.

Electro-chemical power source 82 may be any conventional electro-chemical power source such as a battery. In an embodiment, electro-chemical power source 82 is a disposable battery such as an alkaline battery. Electro-chemical power source 82 provides energy to charge or keep charged rechargeable power source 24 of implantable medical device 16.

Energy from electro-chemical power source 82 is coupled to charging circuit 84 and then to primary coil 86. Charging circuit 84 is conventional serving to modulate an energy signal supplied to primary coil 86. Charging circuit 84 may be similar to and could be identical to circuitry contained in charging unit 50 illustrated in FIG. 3 or any of a number of conventional charging circuits well known and commonly used for inductive coupled charging. Similarly, primary coil 86 is conventional and serves to transmit inductive energy from primary coil 86 to secondary coil 34 of implantable medical device 16. Primary coil 86 may be similar to and could be identical to primary coil 54 illustrated in FIG. 3 or any of a number of conventional coils well known and commonly used for inductive coupling.

In operation, charging sub-assembly 80 operates to charge, or to maintain the charge of, rechargeable power source 34 of implantable medical device 16. Charging sub-assembly 80 is co-located with implantable medical device 16 with primary coil 86 and secondary coil 34 in a juxtaposed relationship, meaning that primary coil 86 is positioned in the proximity of or close to secondary coil 34 so as to effectuate an efficient transfer of energy from primary coil 86 to secondary coil 34 using inductive coupling. Preferably, primary coil 86 is co-axial with secondary coil 34 and positioned as close to secondary coil 34 as is possible given the restraints of packaging. Ideally, primary coil 86 would be within one to two centimeters of secondary coil 34 co-axially aligned, meaning that not only that the axis of primary coil 86 and the axis of secondary coil 34 are parallel but also that the axis of primary coil 86 is laterally aligned with the axis of secondary coil 34.

Charging sub-assembly 80 operates in a like manner to a conventional transcutaneous charger providing energy to implantable medical device 16 through inductive coupling. Because the energy transfer to implantable medical device 16 is identical to the energy transfer implantable medical device 16 would receive from a transcutaneous charger, no or few modifications or adjustments are necessary to implantable medical device 16 to allow implantable medical device 16 to be efficiently charged, or maintained, by charging sub-assembly 80.

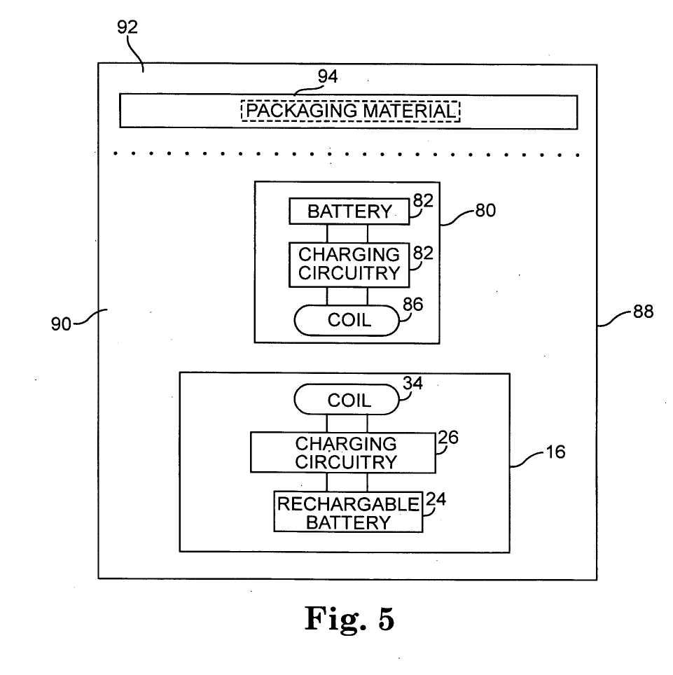

As shown in FIG. 5, charging sub-assembly 80 may be co-located in container 88, which may be a shipping container, storage container or even a temporary holding bin. Container 88 may be a container used to place implantable medical device 16 following manufacture, during shipment to an implant medical facility and/or while awaiting implantation. Container 88 may be conventional in nature intended to securely hold and protect implantable medical device 16, preferably in a sterile environment. Container 88 may be modified in size from a container which would otherwise hold implantable medical device 16 in order to also adequately hold charging sub-assembly 80.

So co-located with primary coil 86 juxtaposed, e.g., co-axially aligned, with secondary coil 34, charging sub-assembly 80 is able to charge or to maintain to the charge rechargeable power source 34 of implantable medical device 16 while implantable medical device 16 awaits implantation, thereby providing an implantable medical device 16 with an appropriately charged, e.g., fully charged, power source 34 when container 88 is opened for implantation of implantable medical device 16. This ensures that patient 18 has an appropriately charged, e.g., fully charge, device 16 eliminates the need of medical personnel to perform an initial charging of implantable medical device 16 immediately prior to implant. This also ensures that implantable medical device 16 may remain in its sterile environment until implantation without the necessity of removing implantable medical device 16 for charging prior to implantation.

Container 88 may have sterile area 90 into which both implantable medical device 16 and charging sub-assembly 80 are co-located. If this is the case, container 88 may also have non-sterile area 92 for holding other material not required, or perhaps not desired, to be sterile, such as packaging material, protective material or literature associated with implantable medical device 16.

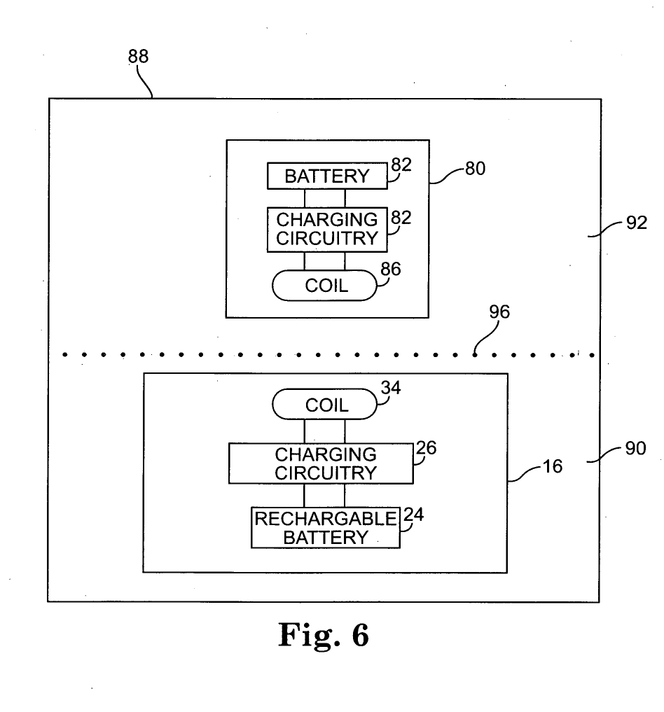

Since charging sub-assembly 80 need not be sterile and since energy transfer to implantable medical device 16 can be accomplished inductively, charging sub-assembly 80 may instead to located in non-sterile area 92 of container 88 (FIG. 6). Primary coil 86 and secondary coil 34 may still be juxtaposed across the sterile—non-sterile boundary 94. This positioning would eliminate the necessity of sterilizing charging sub-assembly 80.

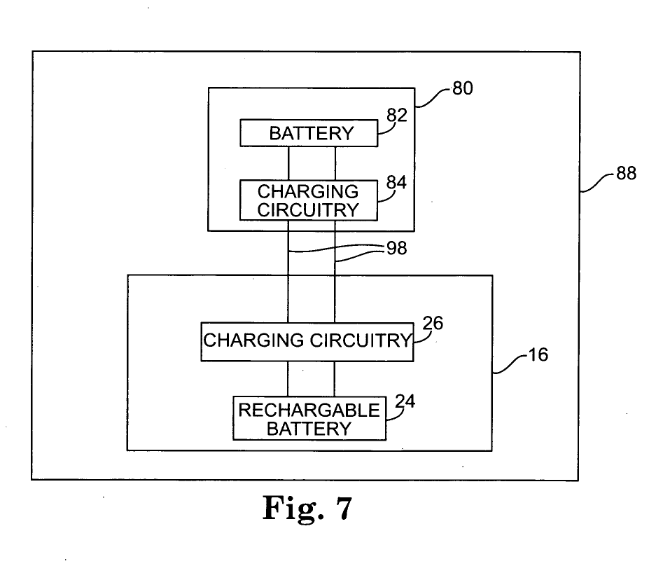

Alternatively, charging sub-assembly 80 may charge, or maintain the charge of, rechargeable power source 34 of implantable medical device 16 by being directly connected to implantable medical device 16 in container 88. In an embodiment illustrated in FIG. 7, charging sub-assembly 80 contains electro-chemical power 82 as in the embodiment illustrated in FIG. 6. Charging sub-assembly 80 also contains charging circuitry 84. Charging circuitry 84 is operatively coupled to hard wire 98, preferably directly, to electronics 26 of implantable medical device 16. Since energy transfer in this embodiment is by hard-wired connection 98, it may not be necessary to modulate the energy signal appearing on hard-wired connection 98 for inductive coupling purposes as in the embodiment illustrated in FIG. 6. Instead, the energy transfer signal provided on hard-wired connection 98 by charging circuitry 84 may be a conventional battery charging signal, perhaps a constant voltage level. Such energy transfer signal can be supplied directly to electronics 26 of implantable medical which may be conventional in nature for receiving a charging voltage or current level from an outside source. Since implantable medical device 16 may otherwise be configured for direct charging because the vehicle for charging of implantable medical device 16 following implantation may be by inductive coupling, electronics 26 of implantable medical device 16 may be modified to accept the direct charging signal on hard-wired connection 98.

It is recognized that implantable medical device 16 could be shipped or stored fully discharged, partially charged or essentially fully charged. Charging, full or partial, could still be desired, as for example, topping off the charge of implantable medical device 16 before implantation.

It is also recognized and understood that secondary coil 34 could be separate from, completely apart from or only semi-attached to the remainder of implantable medical device 16. In this case, the positioning and indicia 130 would still apply to the portion of the apparatus or assembly containing secondary coil 34 or to secondary coil 34 separately.

Thus, embodiments of the external power source for an implantable medical device having an adjustable magnetic core and system and method related thereto are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A storable implantable medical device assembly, comprising:
a container;
an implantable medical device, comprising:
therapeutic componentry;
a rechargeable power source operatively coupled to said therapeutic componentry; and
a secondary coil operatively coupled to said rechargeable power source;
a charging sub-assembly, comprising:
an electro-chemical power source;
a charging circuit operatively coupled to said electro-chemical power source; and
a primary coil operatively coupled to said charging circuit; and
said implantable medical device and said charging sub-assembly being co-located within said container;
said primary coil of said charging sub-assembly and said secondary coil of said implantable medical device being arranged in said container in juxtaposed relationship with said charging sub-assembly charging said rechargeable power source of said implantable medical device through inductive coupling between said primary coil of said charging sub-assembly and said secondary coil of said implantable medical device.

2. A storable implantable medical device assembly as in claim 1 wherein said implantable medical device is contained within a sterile sub-container within said container.

3. A storable implantable medical device assembly as in claim 2 wherein said charging sub-assembly is located within a non-sterile area of said container.

4. A storable implantable medical device assembly as in claim 1 wherein said electro-chemical power source is a battery.

5. A storable implantable medical device assembly as in claim 4 wherein said battery comprises a disposable battery.

6. A storable implantable medical device assembly as in claim 5 wherein said disposable battery comprises a plurality of alkaline batteries.

7. A storable implantable medical device assembly, comprising:
a container;
an implantable medical device, comprising:
therapeutic componentry; and
a rechargeable power source operatively coupled to said therapeutic componentry;
a charging sub-assembly, comprising:
an electro-chemical power source; and
a charging circuit operatively coupled to said electro-chemical power source;
said implantable medical device and said charging sub-assembly being co-located within said container;
said charging circuit of said charging sub-assembly being operatively coupled to said chargeable power source within said container to charge said rechargeable power source while said implantable medical device remains in said container.

8. A storable implantable medical device assembly as in claim 7 wherein said charging circuit is directly connected to said rechargeable power source through a hard-wired connection.

9. A shipping container for an implantable medical device having therapeutic componentry, a rechargeable power source operatively coupled to said therapeutic componentry and a secondary coil operatively coupled to said rechargeable power source, comprising:
a container;
a charging sub-assembly having an electro-chemical power source, a charging circuit operatively coupled to said electro-chemical power source and a primary coil operatively coupled to said charging circuit;
said container being arranged to be able to co-located said implantable medical device and said charging sub-assembly within said container during shipment;
said primary coil of said charging sub-assembly and said secondary coil of said implantable medical device, when so co-located, being arranged in said container in juxtaposed relationship with said charging sub-assembly charging said rechargeable power source of said implantable medical device through inductive coupling between said primary coil of said charging sub-assembly and said secondary coil of said implantable medical device.

10. A shipping container as in claim 9 wherein said implantable medical device is contained within a sterile sub-container within said container.

11. A shipping container as in claim 10 wherein said charging sub-assembly is located within a non-sterile area of said container.

12. A shipping container as in claim 9 wherein said electro-chemical power source is a battery.

13. A shipping container as in claim 12 wherein said battery comprises a disposable battery.

14. A shipping container as in claim 13 wherein said disposable battery comprises a plurality of alkaline batteries.

15. A shipping container for an implantable medical device having therapeutic componentry and a rechargeable power source operatively coupled to said therapeutic componentry, comprising:
a container;
a charging sub-assembly having an electro-chemical power source and a charging circuit operatively coupled to said electro-chemical power source;
said container being arranged to be able to co-locate said implantable medical device and said charging sub-assembly within said container during shipment;
said charging circuit of said charging sub-assembly being operable to charge said rechargeable power source while said implantable medical device remains in said container.

16. A shipping container as in claim 15 wherein said charging circuit is directly connected to said rechargeable power source through a hard-wired connection.

17. A method of storing an implantable medical device having therapeutic componentry and a rechargeable power source operatively coupled to said therapeutic componentry, comprising:
co-locating said implantable medical device and a charging sub-assembly in a single a container; and
operatively charging said rechargeable power source of said implantable medical device with said charging sub-assembly in said container.

18. A method as in claim 17 wherein said charging sub-assembly contains an electro-chemical power source as a source of power to charge said rechargeable power source of said implantable medical device.

19. A method as in claim 18 wherein said electro-chemical power source is a battery.

20. A method as in claim 17 further comprising the step of shipping said implantable medical device in said container while said rechargeable power source is being recharged by said charging sub-assembly.

21. A method as in claim 20 further comprising the step of disposing of said charging sub-assembly following said shipping step.

22. A method as in claim 17 further comprising the step of operatively coupling charging sub-assembly with said rechargeable power source of said implantable medical device through a hard-wired connection.

23. A method as in claim 17 wherein said implantable medical device further has a secondary coil operatively coupled to said rechargeable power source, wherein said charging sub-assembly further has a secondary coil and wherein said co-locating step further comprises arranging said implantable medical device and said charging sub-assembly in said container with said primary coil of said charging sub-assembly and said secondary coil of said implantable medical device in a juxtaposed relationship.

24. A method as in claim 23 wherein said charging step further comprises charging said rechargeable power source of said implantable medical device through inductive coupling between said primary coil of said charging sub-assembly and said secondary coil of said implantable medical device.
